(19) United States
(12) Patent Application Publication
(10) Pub. No.: US 2024/0102954 A1
(43) Pub. Date: Mar. 28, 2024

(54) Identification of Components in a Fluid Flow Using Electrochemical Impedance Spectroscopy

(71) Applicant: Hypersoniq B.V., Capelle aan de IJssel (NL)

(72) Inventors: Lazlo Kleczewski, Capelle aan de Ijsse (NL); Sven Eric Haitjema, Capelle aan de Ijsse (NL); Majid Ahmadi, Groningen (NL); Peyman Tahery, Delft (NL)

(21) Appl. No.: 18/266,878

(22) PCT Filed: Dec. 13, 2021
(86) PCT No.: PCT/EP2021/085571

§ 371 Date: Jun. 13, 2023

(30) Foreign Application Priority Data
Dec. 14, 2020 (EP) 20213704.8

(51) Int. Cl. G01N 27/02; G01N 33/18

(52) U.S. Cl. CPC G01N 27/026; G01N 33/1813

(57) ABSTRACT

Provided is a method and apparatus for the identification of one or more liquid and/or gaseous components in a fluid using Electrochemical Impedance Spectroscopy with a wide range of frequencies. In the preferred embodiment the method of measuring the concentration and/or constituents of a sample including heavy metal ions, uses two or more frequency bands. The measurements from the first frequency band are combined with measurements in the second frequency band such that the concentration of a certain constituent is established in real time (or near real time). A-priori knowledge is used in the combination, and the a-priori knowledge is related to measurements of certain materials at earlier times.

### CROSS REFERENCE TO RELATED APPLICATIONS

This application is the United States national phase of International Patent Application No. PCT/EP2021/085571 filed Dec. 13, 2021, and claims priority to European Patent Application No. 20213704.8 filed Dec. 14, 2020, the disclosures of which are hereby incorporated by reference in their entireties

### BACKGROUND OF THE INVENTION

### Field of the Invention

The present disclosure is related to a method and apparatus for the identification of one or more liquid and/or gaseous components in a fluid using Electrochemical Impedance Spectroscopy with a wide range of frequencies. At a later stage the frequencies can also include the higher ranges, e.g., infrared, ultraviolet, Xray etc.

### Description of Related Art

For various situations there is the need to identify the composition of unknown substances, varying from chemical process control to forensics. Various methods are in existence that can be used to identify compounds or substances, many of them mainly suitable for laboratory situations. Many sensors can only detect small number of compounds.

Electrochemical Impedance Spectroscopy (EIS) can be used over a very wide detection range, currently in laboratory environments. Due to the ambiguity of the data obtained by this method, it is only suitable for detecting changes in concentrations of known particles, or for comparison to known references.

### SUMMARY OF THE INVENTION

The present patent disclosure provides a method of measuring the concentration and/or constituents of a sample by electrochemical impedance spectroscopy (EIS) in frequency ranges results in particular impedance values, which are dependent on a certain constituent and its concentration at a specific frequency established in real time (or near real time).

Preferably in the method a sweep over the different frequency bands takes place.

Preferably a rinsing takes place after each measurement.

Preferably also a priori knowledge is used in applications wherein the compounds are known to a certain extent and the concentrations have to be monitored.

Preferably the a-prior knowledge is related to measurements of certain materials at earlier times; such information can be stored in memory.

In a set-up wherein the constituents are known per se and are dissolved in a known solvent, such as water, the concentrations are preferably measured around a peak and/or valley marking point in the Bode plot of at least one frequency band.

Typically, the real part (i.e., the resistivity) of the Bode plot shows a peak in a range where certain solutions with constituents therein show a space charge polarization, such as in a frequency range at the lower frequencies, such as 0.1-100 Hz. The frequencies are therefore preferably sufficiently distanced from each other, so that also other phenomena at very different frequencies are observed in the Bode plot.

At much higher frequencies such as in the range of 0.5-2 GHz in many instances a valley can be detected in the real part of the Bode plot due to Ionic relaxation and dipolar relaxation of compounds.

By combining different parts of the spectrum an unknown constituent in a known solvent can be detected unambiguously. Also, parts showing changes of the complex part (i.e., capacitance) of the Bode plot can be used.

Preferably the frequency bands comprise frequencies from 0.1 Hz-30 GHz, preferably 10-100 kHz, 100 kHz-1 MHz, and/or 1 Mhz-1 GHz.

The present patent disclosure also provides an apparatus, comprising:
a first module configured to provide voltage and/or electrical currents in a first frequency band and to measure the impedance of the sample in the first frequency band,
a second module configured to provide voltage and/or electrical currents in a second frequency band different from the first band and to measure impedance in the second band.

Preferably the apparatus comprises more than three, preferably 3-12 modules for different frequency bands, ranging from 0.1 Hz-10 GHz.

Using this apparatus, the measurements in different frequency bands can be executed (almost) simultaneously making (near) real time applications in monitoring and control feasible.

Preferably the apparatus comprises a housing, wherein the modules are arranged, as well as a system controller, data processor, power module, and/or one or more environmental sensors; the temperature in the housing preferably sufficiently controlled for reproducible measurements. The apparatus can be placed in the flow of wastewater of an industrial or harbor site, or in a bypass of the main flow.

In this way such apparatus may also become mobile, which will be very useful for certain applications.

Preferably the apparatus is provided with a heating/cooling unit connected to a supply unit for providing cooling/heating fluid, and more preferably each module comprises a board provided with a temperature sensor connected to a secondary heating/cooling element for finely controlling the temperature of the EIS module.

The apparatus can be provided with AI (Artificial Intelligence) by using old measurements from data storage for learning purposes, or also extrapolation of unknown data.

In this preferred embodiment the measurements will be independent of environmental conditions (temperature, vibrations, light etc.) as much as possible.

Further advantages, features and details of the present patentable subject matter will become apparent from the following description with reference to a drawing, in which show:

### BRIEF DESCRIPTION OF THE DRAWINGS

FIG. 1 is a block diagram of a preferred embodiment of a sensor system according to the present disclosure;

FIG. 2 is a block diagram of a preferred sensor block of the embodiment of FIG. 1;

FIGS. 3A-C are Bode Plots of measurements obtained by the preferred embodiment of FIG. 1;

FIG. 4 is a Bode Plot of the impedance of characteristic point;

FIGS. 5 and 6 are Bode plots of two solutions at different temperatures;

FIGS. 7A and 7B are plots of a solution with alloys dissolved in water;

FIG. 8 is a scheme explaining the way to identify an unknown material using the preferred embodiment of FIG. 1 using 13 measurements;

FIGS. 9A, 9B and 9C are respectively a top view, a side view and a perspective view of a detail of a design of an electrode;

FIG. 10 is a graph comparing the sensitivity of different materials used and cell designs, in terms of impedance changes;

FIG. 11 is a diagram of another example of an apparatus according to the present disclosure;

FIG. 12 is a diagram of a detail of FIG. 11;

FIGS. 13A and 13B are diagrams explaining the operation of the apparatus of FIGS. 11 and 12;

FIG. 14 is a diagram of a detail of FIG. 11;

FIG. 15 is a diagram of a detail of FIG. 12;

FIG. 16 is a graph of concentration measurement of Zinc Sulphate in water;

FIG. 17 is a graph of concentration measurements of Lead Nitrate in water; and

FIGS. 18 and 19 are graphs of two mixed solutions of Pb and Zn of 5 ppm and 50 ppm, respectively.

### DESCRIPTION OF THE INVENTION

In what follows, the terms “Electrochemical Impedance Spectroscopy” and “EIS” should be treated as synonyms.

In what follows, the term “Water Treatment” designates any process that improves the quality of water to make it more acceptable for a specific end-use. The end use may be drinking, industrial water supply, irrigation, river flow maintenance, water recreation, or many other uses, including being safely returned to the environment. To conduct proper water treatment, detecting the toxic compounds and particles in the water is an important step. Among these contaminants, heavy metal ions (HMI) are considered highly toxic at trace levels and can lead to various diseases after being consumed or absorbed by human beings. The emission of heavy metal into the environment can result from both natural and anthropogenic activities. However, the major emission occurs during the mining operation and industrial processes. Additional sources of Heavy Metal Ions are the exhaust of automobiles and household waste disposal.

As “Heavy metal contamination” is considered contamination by any group of metals or metalloids with atomic weights between 63.5 and 200.6 g/mol and possesses a density greater than 4 g/cm3, or five times greater than water. By this definition, more than 50 elements in the periodic table can be classified as heavy metals. However, the term “heavy metal” is more commonly referred to as the metallic/semi-metallic elements that pose a threat to human health and flora and fauna in the environment due to their chemical properties and accessibility. This definition, concerning the toxicity, thus narrows down the categories of heavy metal to 17 elements. These elements, generally including mercury (Hg), cadmium (Cd), arsenic (As), chromium (Cr), lead (Pb), zinc (Zn), copper (Cu), iron (Fe), silver (Ag), and nickel (Ni), have adverse effects on living organisms when being entered the body at a certain level.

The toxicity of these elements to the human body comes from the inhibition of enzymes and the induction of oxidative stress. To ensure society's safety, various agencies at the national, regional, and international levels have set the concentration limits of heavy metal in the drinking water, such as the ones referred to at Guidelines for Drinking-Water Quality (GDWQ) 4th edition published by WHO in 2011.

Electrochemical impedance spectroscopy (EIS) is a technique that investigates the dielectric properties of a physical system. Due to its simplicity and versatility, EIS is widely used in the food industry to examine the concentration of bacteria, the composition and quality of food, in the biomedical field to reveal information about the interactions between biomolecules, in materials science for the qualitative evaluation of coatings, nanocomposite synthesis and film formation.

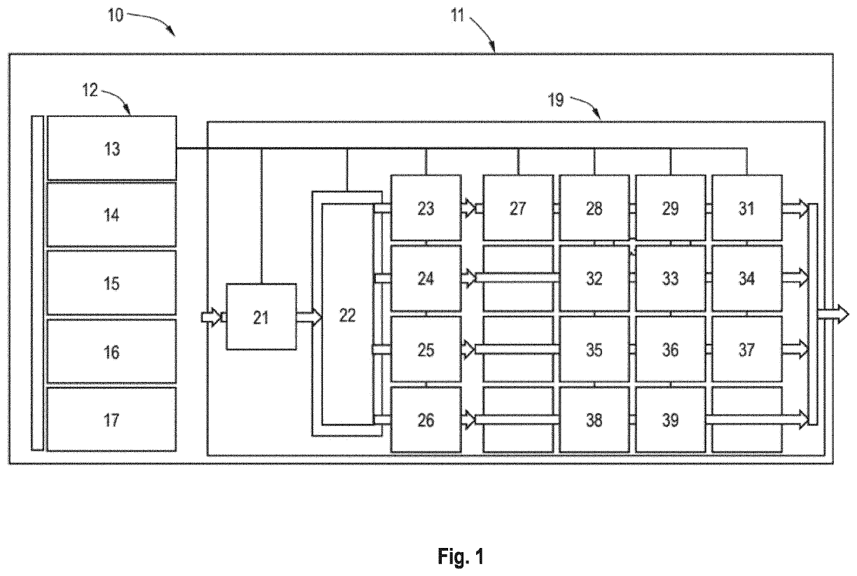

A preferred embodiment of a system 10 (FIG. 1) comprises in a schematic form a housing 11, in which a main board 12 is arranged on which a system controller 13, a data processor 14, a communication unit 16, a power unit and environmental sensors 18 are mounted.

In the housing 11 also a manifold on the backplate 19 is provided. Cooling/heating fluid is supplied by a pump 21 through a manifold element 22 to valve elements 23, 24, 25 and 26, resp. On the back plate 19 there are arranged twelve EIS measuring units 23-39 each configured to execute measurements in different frequency ranges or bands, viz. unit 23 from 0.1-1 Hz, unit 28 from 1-10 Hz, unit 29 10-100 Hz, unit 31 from 0.1-1 kHz, unit 32 from 1-10 kHz, unit from 10-100 kHz, unit 34 from 0.1 MHz-1 MHz, unit 35 from 1-10 MHz, unit 36 from 10-100 MHz, unit 37 from 0.1-1 GHz, unit 38 from 1-10 GHz and unit 39 from 10-100 GHz. On the backplate four further spaces are available to mount further measuring modules.

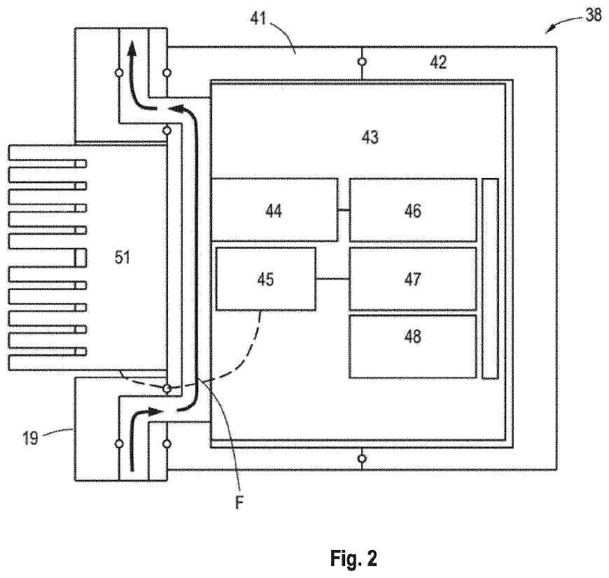

Each sensor module, e.g. 38 (FIG. 2) comprises a housing 41, provided with a cover 42, preferably of metal (such as to form a Faraday shield against EM-waves), wherein a sensor board 43 is disposed. On the sensor board there are mounted an EIS sensor 44, temperature sensor 45, an EIS controller 46 and fine-tuning temperature controller 47 and a communication unit 48.

The cooling/heating fluid F flows from the backplate along the measuring module while heated/cooled by a fine-tuning heating/cooling element, which is electrically connected to the sensor 45 on the board and also to the controller 47 on the board.

Primary temperature control is executed by system controller 13 which is electrically connected to all measuring modules 23-39, to the primary heating/cooling element, as well as to the supply pump. As will be understood the temperature of the measuring modules has to be kept constant as much as possible during the measurement process. For that purpose, usually additional cooling by an element such as 51 will be necessary for the module being active at a certain moment in time.

After each measurement, the system is preferably reset. A probe can be reset by rinsing with water with or without a chemical cleaning agent.

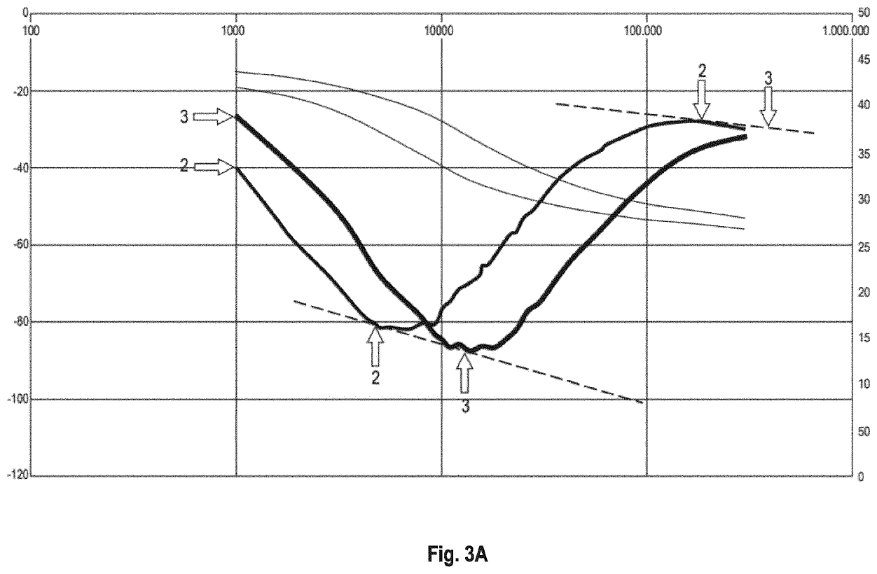

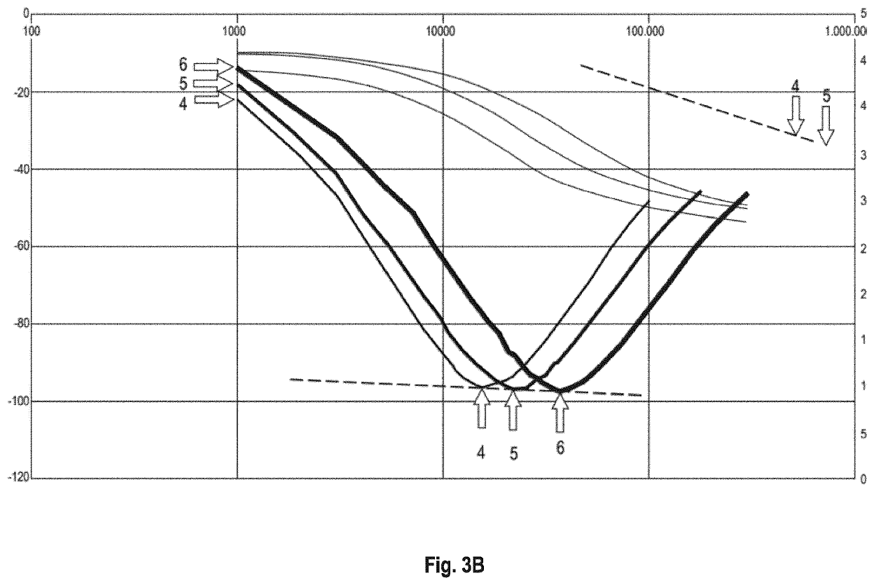

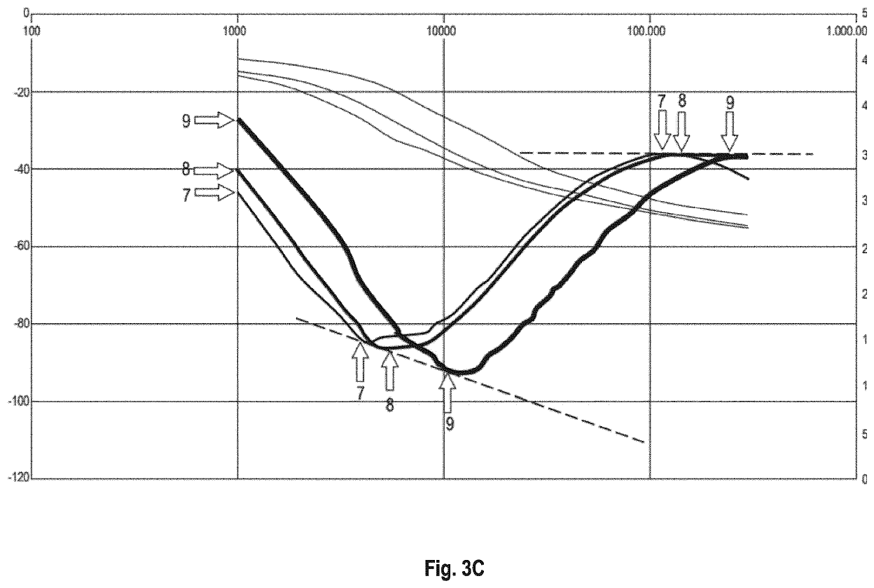

With the sensor system of FIG. 2 a number of measurements were made in a frequency range of 1 kHz-300 kHz; the results are shown in FIGS. 3A-3C. Three salts were dissolved in three different concentrations in demineralized water:
Sample 1 ZnSO₄ (500 ppm in H₂O)—not shown
Sample 2 ZnSO₄ (1000 ppm in H₂O)
Sample 3 ZnSO₄ (2000 ppm in H₂O)
Sample 4 FeNH₄ (SO)₂ (500 ppm in H₂O)
Sample 5 FeNH₄ (SO)₂ (1000 ppm in H₂O)
Sample 6 FeNH₄ (SO)₂ (2000 ppm in H₂O)
Sample 7 Pb(NO₃)₂ (500 ppm in H₂O)
Sample 8 Pb(NO₃)₂ (1000 ppm in H₂O)
Sample 9 Pb(NO₃)₂ (2000 ppm in H₂O)

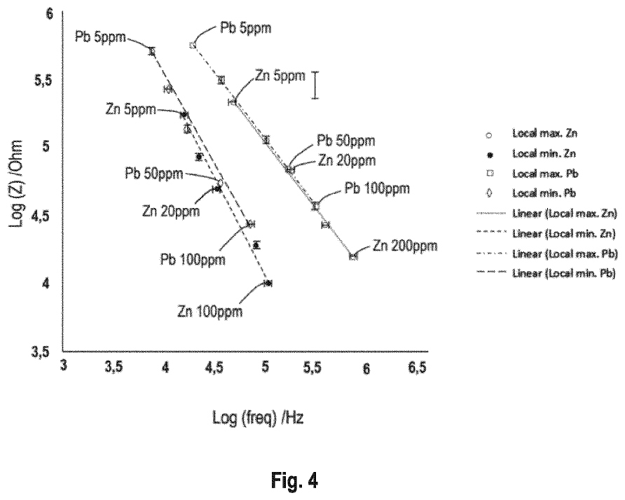

Further measurements were made for solutions of Pb(NO₃)₂ (Pb) and ZnSO₄ (Zn) (see Bode plot of FIG. 4). The values of a local minimum and maximum for different concentration levels 5, 10, 20, 50 and 100 ppm resp. are on a straight line approximately and can be well distinguished from each other for the different concentrations (although Pb 50 ppm and Zn 20 ppm are somewhat close. FIG. 4 also shows the error margins, as the measurement were repeatedly executed.

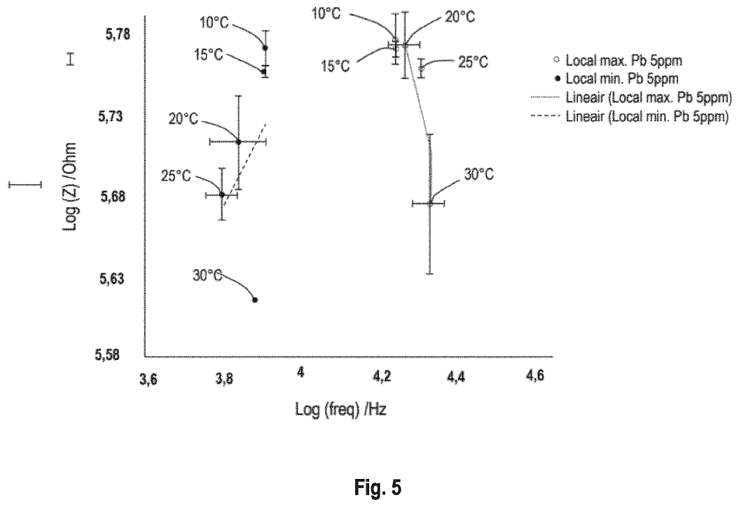

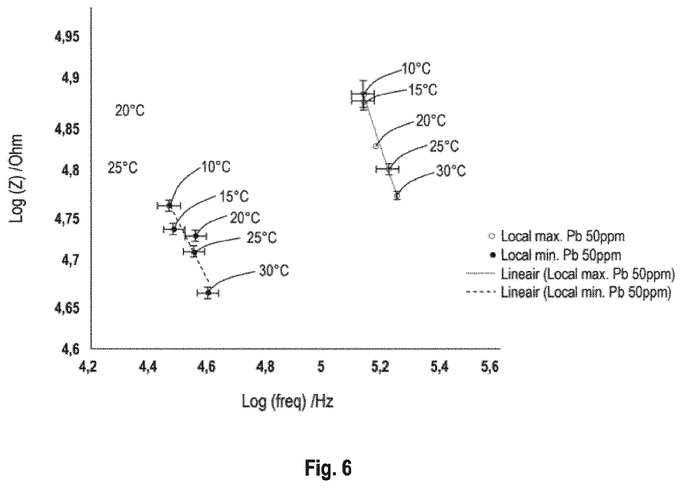

In the Bode plot of FIGS. 5 and 6 the values Pb 5 and 50 ppm are shown for different temperatures. A deviation of 5 degrees Celsius will lead to less than 1% error margin. In a further embodiment the influence of temperature to the measured values is measured and the sensor is calibrated accordingly.

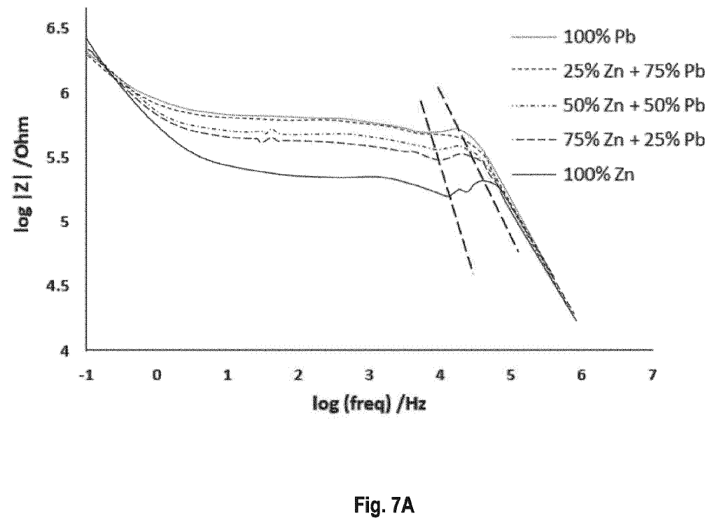

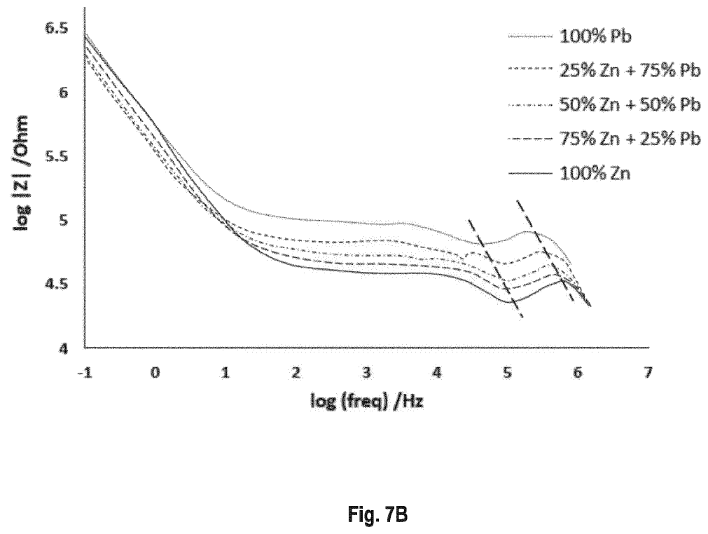

FIG. 7 shows that also mixed solutions with Pb and Zn are distinguishable for the different mixing ratios (at 20 degrees Celsius).

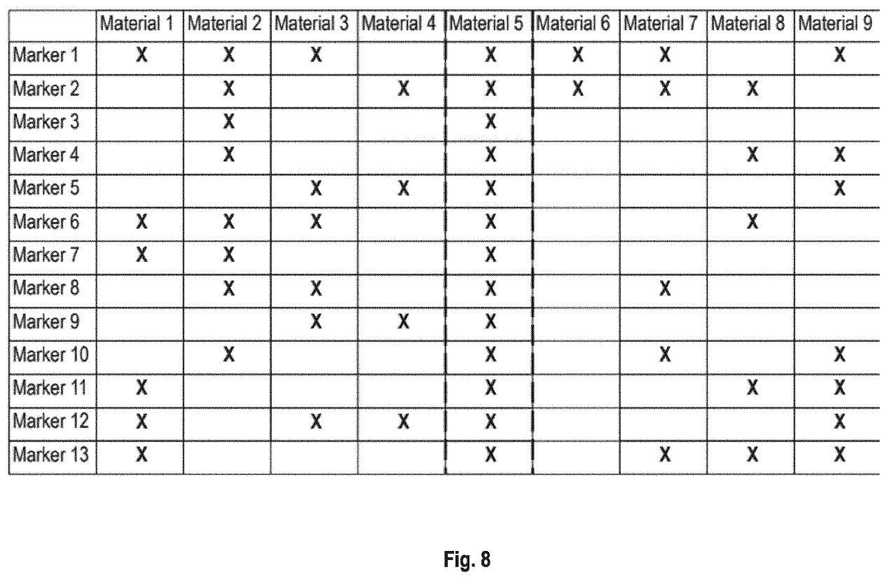

In the apparatus according to the description for instance thirteen marker frequencies could be chosen, markers 1-13 (FIG. 8). By these thirteen marker frequencies a solution among nine materials 1-9 can be unambiguously identified in the right concentrations, i.d. material 5 to which all thirteen measurements correspond.

Data ambiguity is avoided in this way, so that a device is provide that can autonomously identify the composition of unknown substances at real-time or near real-time (within a one or a few seconds).

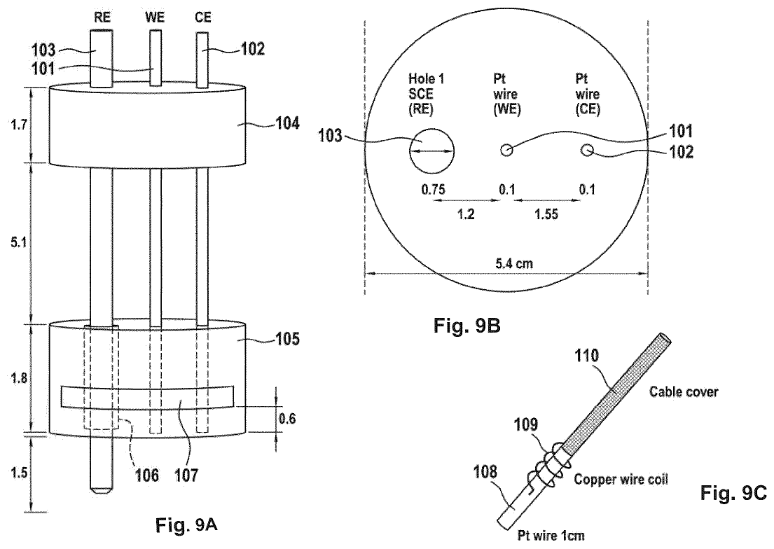

In the preferred embodiment represented in FIG. 9, a three-electrode system was used, which consist of a working electrode or WE 101, a counter electrode or CE 102, and a reference electrode or RE 103. During the EIS measurement, the AC voltage was applied on both WE and CE, while the output signal was measured between RE and WE. Pure platinum (Pt) wire was chosen to be the material for both working (WE) and counter (CE) electrodes in the standard design due to its chemical and electrical properties, whilst for the reference electrode 103, the saturated calomel electrode (SCE) was chosen due to its availability and stability.

Two platinum wires 108, 110 with approximately 1 cm length and 1 mm diameter are connected with copper cable 109 without soldering before being embedded in an acrylic resin holder 105 by cold mounting. During the cold mounting process, ClaroCit powder (dibenzoyl peroxide) and ClaroCit liquid (methyl methacrylat and tetramethylene dimethacrylate) supplied by Struers ApS is taken in a 2:1 ratio and mixed. In addition, an extra plastic rod 106 with 7.5 mm diameter was covered with silicone oil and installed in the setting, parallel to the working and the counter electrodes, which helped create a hole for the insertion of the reference electrode 103.

After the acrylic resin was fully dried in a high-pressure environment to prevent the formation of air bubbles, the bottom of the holder was sanded with SiC sand-papers, with the numbers of P80, P180, P320, P800, P1200, P2000. After grinding, the bottom of the holder was polished with fine diamond particles with the size of 3 μm and 1 μm until a mirror-like surface was reached. After polishing and before the EIS measurements, the acrylic resin holder was finally cleaned with deionized water and isopropanol and dried with an air gun. The Reference Electrode 103 is composed of a Pt rod of a maximum diameter of 10 mm, which has a first exposed part 108, a second part which is surrounded by a coper wire coil 109 and a third part which is covered by a metal foil 110. The Reference Electrode 103 is protruding from the bottom surface of the acryl resin holder by at least 1 cm, preferably by 1.5 cm. Optionally, an additional holder (104) of similar geometry can be provided closer to the connection of the electrodes to the cables.

This kind of design makes sure that the distance between the working and the counter electrodes is fixed at all times. It is worth noting that the distance between each electrode was set bigger than 1 cm to reduce the effect of stray capacitance, which may result from the storage of the electric charge between platinum/copper wires. In addition, the distance between the WE and the RE was held closer than that between the WE and the CE. This design aims to decrease the ohmic losses due to the residual solution between the WE and RE. Another parameter to be fixed is the dipping depth of the electrodes into the solution, which is between 0.4 and 0.8 cm, preferably 0.6 cm. An easy way of marking the dipping depth is by marking the position with a marker 107, such as a tape.

According to a second embodiment of the electrodes system of the present disclosure, the connection between the copper wire and the platinum wire was made by soldering with tin, in order to eliminate the possibility that the inductive behavior in the EIS result comes from the copper coil at the connection point. Another difference between the standard design and the first modified design is the distance between electrodes.

According to another embodiment of the present disclosure the saturated calomel electrode of the electrodes system is replaced by yet another platinum wire. This replacement of the material of the reference electrode made clear that the behavior of the EIS sensor did not change. Using the same materials for all three electrodes makes it easier to fabricate the EIS sensor in the form of chips. The mass production of the sensor chip can be realized by depositing the desired materials on a wafer and cut it into pieces.

According to yet another embodiment of the present disclosure the three electrodes are forming a triangle with the distance between the working electrode 101 and the reference electrode 103 being approximately 1.2 cm, between the working electrode and the counter electrode being approximately 1.5 cm and between the counter and the reference electrodes approximately 1.75 cm. Moreover, the working electrode (101) is replaced by a non/conductive material recovered by removable platinum thin foil of approximately 1.2×1.2 cm surface area and thickness between 0.10 mm and 0.15 mm. The exposed area of the Pt film can be of circular form of 0.1 cm diameter or a square of 1 cm×1 cm or of any other form.

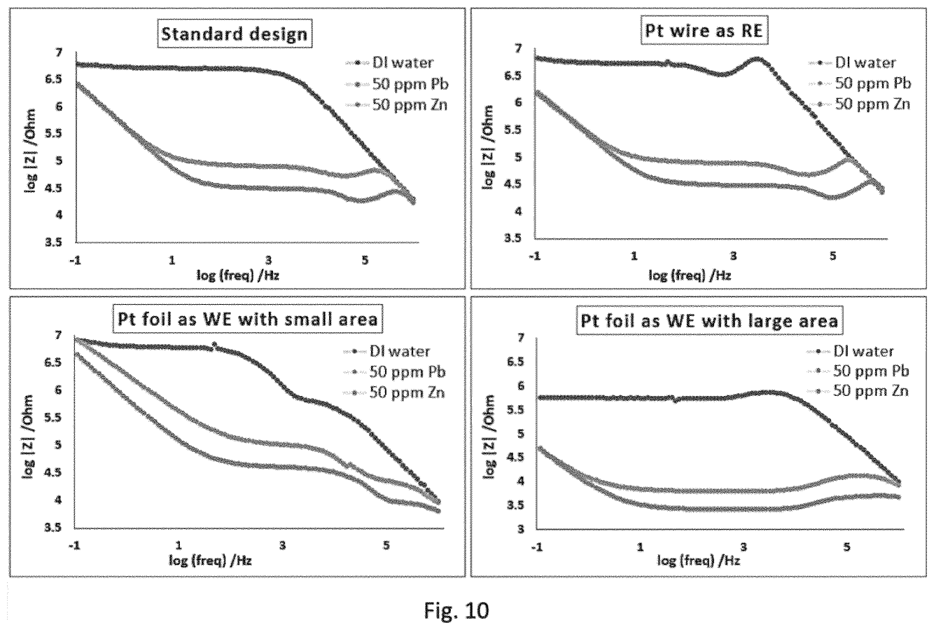

FIG. 10 compares the sensitivity of different cell designs, in terms of impedance changes in log scale compared to deionized water (DI) at 317 Hz. The result shows that the impedance changes of the standard design, soldering modification, and the design with Pt wire as RE are similar.

In FIG. 10 again, the impedance changes for the design of “Pt foil as working electrode with a small area,” are less than the impedance changes of the standard design, indicating lower sensitivity in response to the presence of HMIs. However, the decrease of impedance changes of this design is partly because the plateau region of DI water and HMI solutions do not align well at 371 Hz.

We conclude that the design of “Pt foil as WE with a large area” the results of which are represented at the bottom right of FIG. 10, the impedance changes are the highest among all the cell designs, indicating the highest sensitivity in response to the presence of HMIs. It should be noted that the sensitivity mentioned in this section refers to the degree of impedance change compared to DI water when there are HMIs present in the solution. Higher sensitivity indicates a larger extent of impedance change. However, for the design of “Pt foil as WE with a large area,” the detection of HMI concentration can become more limited than the other designs when the overall impedance value of the solution is low. This happens when the concentration of ions inside the solution are high, since it is more difficult to distinguish the characteristic points for a specific HMI solution when the overall impedance of the system is high.

Using the EIS technique, a few molecules with a large dipole effect can have the same result as a large number of molecules with a small dipole effect. Similar effects are present at other mechanisms EIS can detect, like the ion-relaxation, Space Charge Polarization or conductive regions.

Since the molecules of the substance to be measured affect the different EIS mechanisms in a different way, e.g. a large heavy molecule, with a small dipole, will have a larger effect on the Space Charge Polarization, with a smaller effect on the dipole relaxation. While a light molecule with a large dipole, will show the opposite behavior. Whereby each EIS region will show similar mechanism. This difference makes it possible to positively recognize the substances even if at one or more of the regions the measured results are similar.

Considering a priori knowledge of how individual substances or compounds behave in the different EIS regions, and a priori knowledge how combinations of compounds influence the measurements, by comparing these with measurements of an unknown substance, it becomes possible to deduct which substance and compounds fit the measurement and thus what the composition of the unknown substance must be.

This a priori knowledge could for example be how each of certain points in the Bode plot change in relation to substance composition, or concentration.

Further analysis of the data, like for example by looking at the real and imaginary signal parts, may yield additional points, or e.g. by using the complete data set as a fingerprint.

Once the substance is and its compounds are known, using a similar method, the concentration of each compound can be deducted by comparing the measured results to prior knowledge of the EIS behavior with different concentrations.

Further data analysis has shown that each compound can be detected independently, as the spectra of different constituents are superposed on each other.

By combining an EIS sensor with an algorithm for the substance identification, it is possible to autonomously identify materials in a real-time setting and allow immediate acting upon this identification.

As expected from literature samples 1-3 show measured minimum points on an (approximate straight line, as do samples 4-6 and samples 7-9. The measurements correspond with the theory and were also confirmed with an apparatus with limited frequency capabilities; in this respect it is herewith emphasized that the claims are not limited by any theory.

Furthermore, the theory learns that the different molecules (big/small, heavy/light, small/large dipole effect) of different substances will have a on The Space Charge Polarisation and a different effect on the dipole relaxation. Therefore, the different EIS regions can each make a different positive assessment of the molecules present.

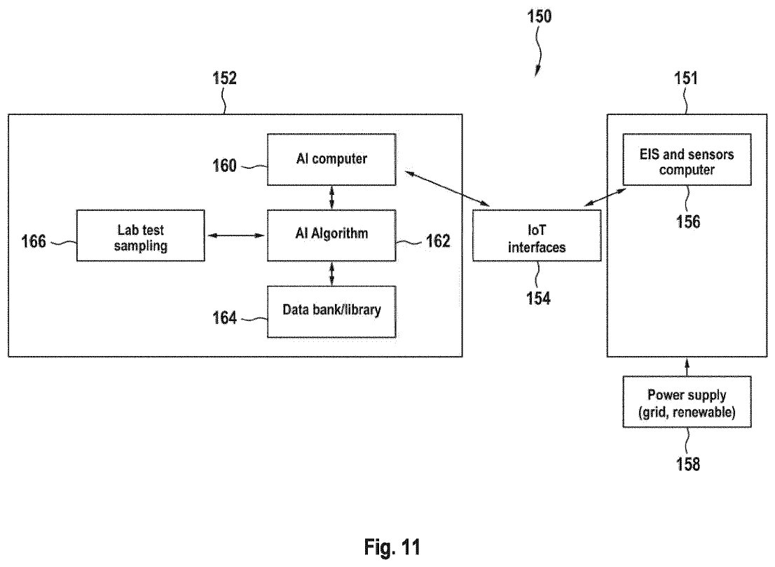

In a preferred set up of FIG. 11 the apparatus 150 comprises a measuring part 151 and a processing part 152. The parts are connected through interfaces 154 with the Internet (of Things).

The measuring part 151 comprises a computer and EIS and sensor parts 156. The measuring part is connected to a power supply, either connected to the grid or provided with a battery (renewable), or both. The measuring part cab be located close to the processing part; more typically the measuring part is located remotely, viz. anywhere in the world where there is Internet available.

The processing part 152 comprises an AI computer 160 provided with an AI algorithm and connected to a data bank/library wherein the a priori knowledge of earlier measurement is stored, AI standing for Artificial Intelligence The processing part is typically located near a laboratory so that Lab test sampling data 166 can be added to the AI algorithm and databank/library.

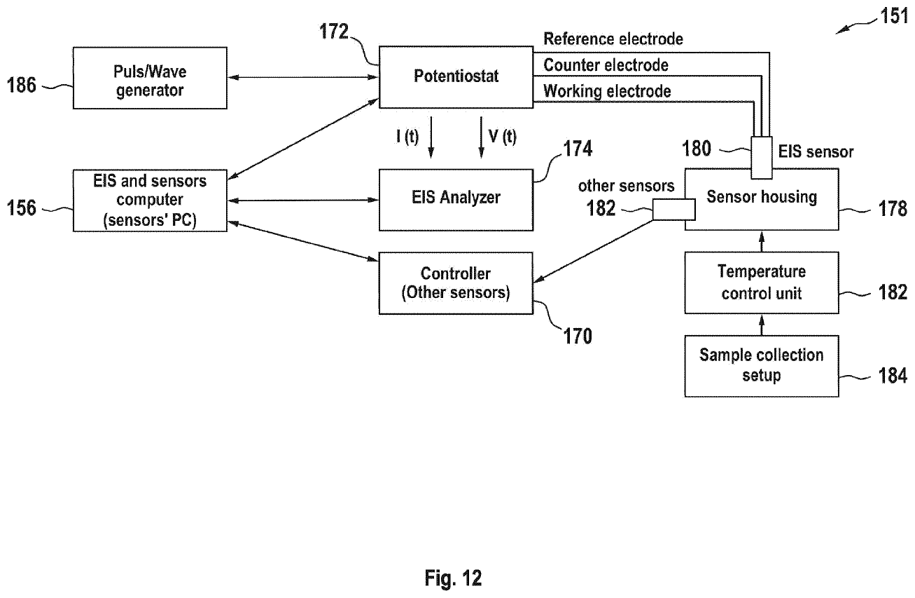

The measuring part 151 (FIG. 12) comprises the computer 156 which is connected to a controller 170 for other sensors, a potentiostat 172 and an EIS Analyzer 174. Samples are measured in a sensor housing provided with a EIS sensor 180 of which a reference electrode, a counter electrode and a working electrode are connected to the potentiostat 176. The sensor housing is also provided with other sensors, such as temperature sensors which are connected to the controller 170 which drives a temperature control unit to control the temperature also for a sample collection setup.

The potentiostat is also connected to a pulse wave generator 186 for providing waves from less than 1 Hz to 100 MHz are even GHz. The potentiostat 172 transmits waves in a certain frequency band to the EIS sensor and provides the applied Voltage V(t) and measured current I(t) to the EIS Analyzer 174.

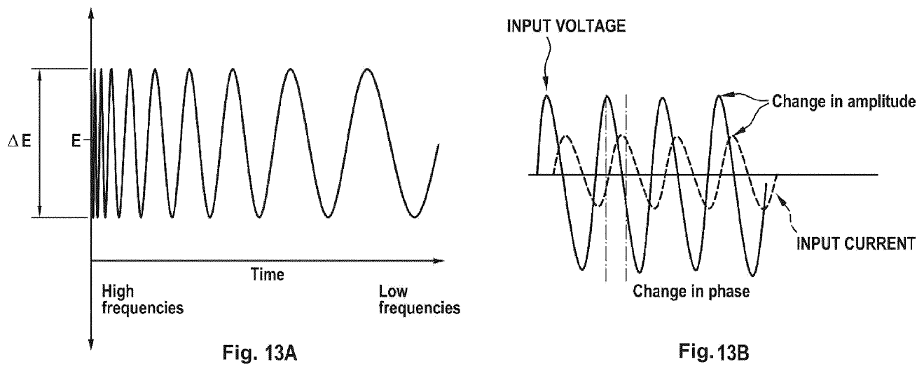

Applied voltage typically swings around an average value E with an amplitude ΔE (FIG. 13A). The amplitude swings faster in time at higher frequencies than at lower frequencies.

The output current usually shows a change in amplitude and in phase for a certain input voltage at a certain frequency (FIG. 13B).

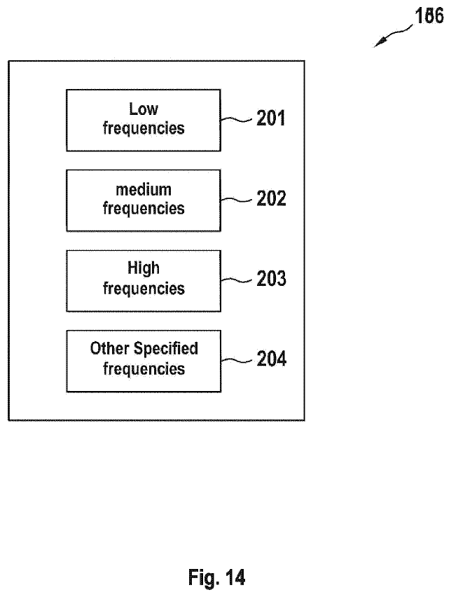

The pull/wave generator 186 (FIG. 14) is preferably provided with a unit 201 for low frequencies, e.g. below 1000 Hz, a unit 202 for medium frequencies, e.g. 1 kHz-50 kHz, and a unit 203 for high frequencies, e.g. above 50 kHz. Preferably also the generator is provided with a special unit 204 for other specified frequencies e.g. dependent on the site, e.g. a refinery, an oil or chemical storage a drinking water facility etc.

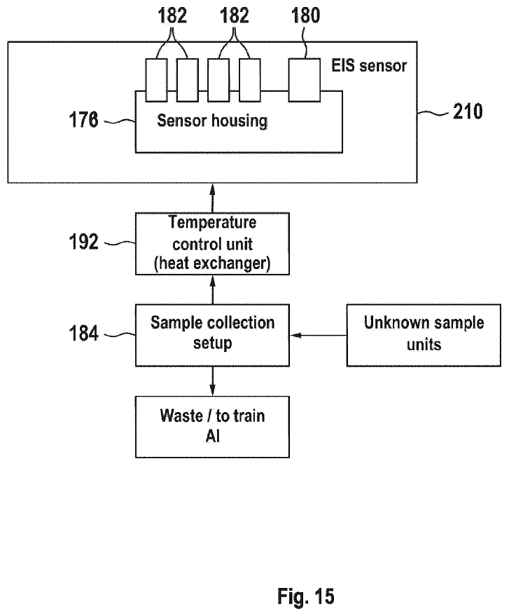

The sensor housing 176 (FIG. 15) having the EIS sensor 180, has four other sensors 182, for temperature, hardness, vibrations, EM field etc. The housing is provided with a shield 210 against mechanical vibrations and also electromagnetic (EM) influences. The temperature control unit is shown to have a heat exchanger to keep the temperature at the desired level independent of the environment (including the time of the year and the position on the earth). The sample collection unit can operate with batches of liquid (also with possibly collection of gas dissolved in the liquid) or with continuous flow of liquid, for which purpose the necessary filters, flowmeters, pressure controllers etc. should be provided.

The sample collection unit 184 can also be provided with unknown samples from unknown sample unit, while waste can also be sent to the laboratory e.g. at the processing location such as to train the AI algorithm and/or load the library with further data and to increase the a priori knowledge in that way.

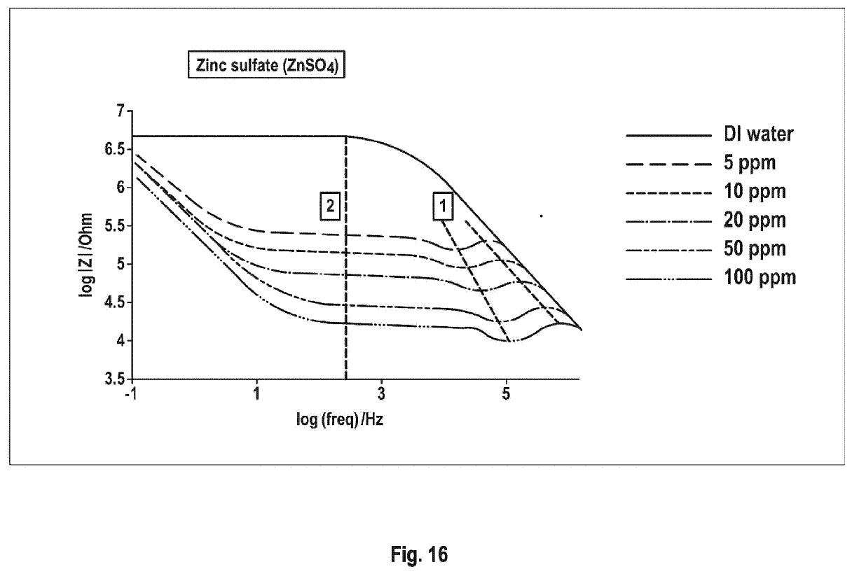

The concentrations of Zinc sulphate (FIG. 16) as measured with the Analyzer and EIS computer show stable values over the range form 0.1 Hz to 1 MHz, even for concentrations as low as 5 ppm.

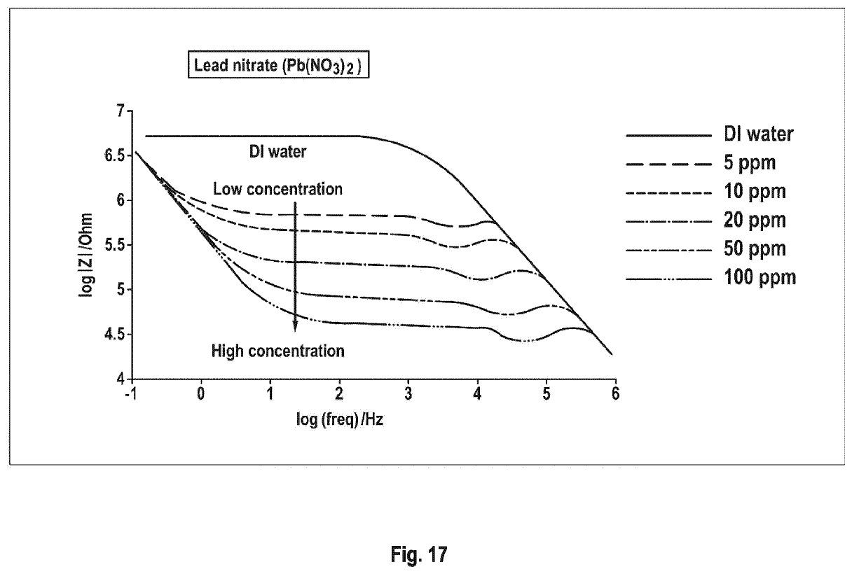

The same holds for Lead nitrate (FIG. 17).

The impedance change relative to (DI) water ranges from 19% (5 ppm) to 36% (100 ppm) lower for Zn ions, and from 13% (5 ppm) to 31% for Pb ions.

To identify ions and to measure concentrations the measuring part on site will use medium to high frequency measurements which can de done in a time period of seconds. If the outcome is not unambiguous the measurements will be sent (over the Internet) to the processing part where the AI algorithm uses the library/databank to analyze the measurements and to send a request automatically to the measuring part to measure again at e.g. a lower frequency (which takes longer). The AI algorithm will be able to combine the measurements with the databank wherein the a priori knowledge is stored and determine the concentration of heavy metals in the sample.

As the processing will be done centrally for a number of remote locations the AI algorithm will be on a steep learning curve, such that more and more samples and will be recognized in a relatively short time period.

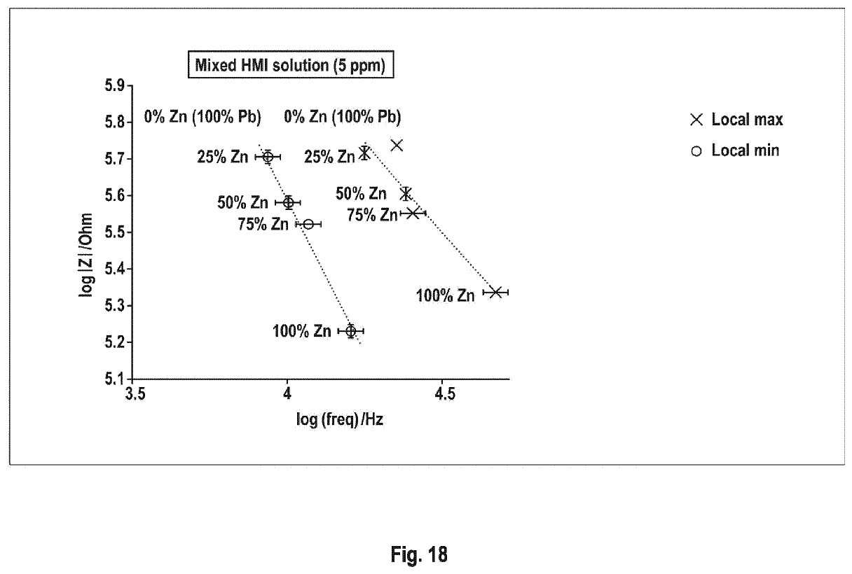

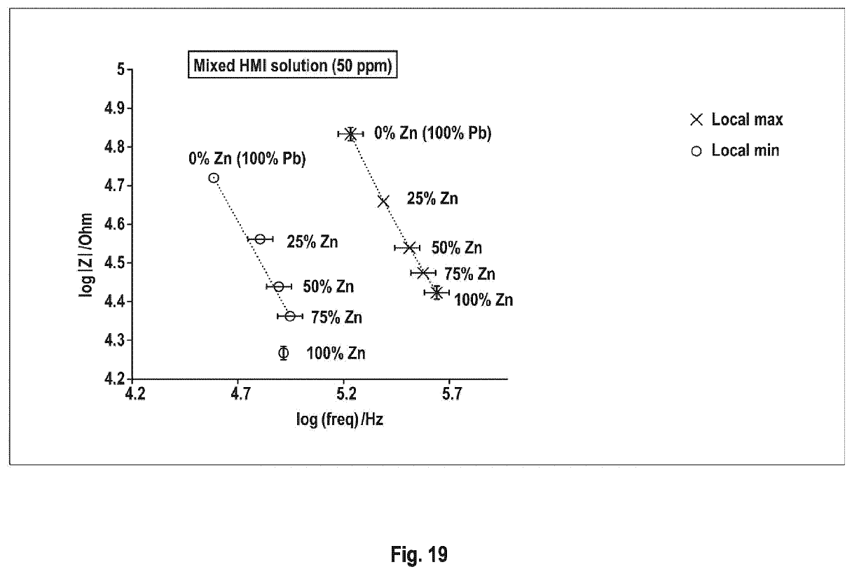

In the example of FIG. 4 showing a ambiguity in a mixed solution the AI algorithm will work with a priori knowledge of FIGS. 18 and 19 wherein the outcome of the mixed solutions for 5 ppm and 50 ppm are measured at the local minima and maxima at around 100 KHz—see the graph of FIG. 16. If necessary, the AI algorithm can order further measurements to be done by the onsite measuring part.

It is the expectation that the present patent disclosure will make a major contribution in monitoring liquid and/or gaseous flows not only for water, e.g. in industrial sites but also in hydrocarbon applications as well as in other industries such as food where contaminants are undesirable, bridging the gap between laboratory and real life applications.

The present patent disclosure is not limited to the description, theory and embodiments above; the requested rights are determined by the following claims, within the scope of which many modifications are feasible.

The scope of the present patent disclosure is also determined by the embodiments or examples according to the following clauses:
1. Method of measuring the concentration and/or constituents of a sample by electrochemical impedance spectroscopy (EIS) in two or more frequency bands, wherein the measurements from the first frequency band are combined with measurements in the second frequency band such that the concentration of a certain constituent is established in real time (or near real time).
2. Method of clause 1, wherein a priori knowledge is used in the combination.
3. Method of clause 2, wherein the a priori knowledge is related to measurements of certain materials at earlier times.
4. Method of clause 1, 2 or 3, wherein the constituents known per se are dissolved in a known solvent, such as water, and the concentrations are measured around a peak and/or valley marking point in the Bode plot of at least one frequency band.
5. Method of clause 1, 2 or 3, wherein an unknown constituent in a known solvent is detected unambiguously.
6. Method of any of clauses 1-5, wherein the frequency bands comprise frequencies from 0.1 Hz-30 GHz, preferably 10-100 kHz, 100 kHz-1 MHz, and/or 1 Mhz-1 GHz.
7. Method of any of clauses 1-6, wherein the frequency is swept over the different frequencies.
8. Method of any of clauses 1-7, wherein rinsing takes place after each measurement.
9. Apparatus, comprising:
   a first module including a communication unit and configured to provide voltage and/or electrical currents in a first frequency band and to measure the impedance of the sample in the first frequency band;
   a second module including a communication unit and configured to provide voltage and/or electrical currents in a second frequency band different from the first band and to measure impedance in the second band.
10. Apparatus according to clause 9, comprising 12 modules each module functioning in a different frequency band, ranging from 0.1 Hz-10 GHz.
11. Apparatus according to clause 9 or 10, comprising a housing, in which the modules are arranged, as well as a system controller, data processor, power module, and/or one or more environmental sensors.
12. Apparatus according to clause 9, 10 or 11, provided with a heating/cooling unit connected to a supply unit for providing cooling/heating fluid.
13. Apparatus according to any of clauses 9-12, wherein each module comprises a board provided with a temperature sensor connected to a secondary heating/cooling element for finely tuning the temperature control of the EIS module.
14. Apparatus according to any of clauses 9-13, provided with memory for storing data from earlier measurements, either locally or, preferably, remotely in a network with on-line access.
15. Apparatus according to any of clauses 9-14, provided with computer power for AI, or deep learning.
16. Apparatus according to any of clauses 9-15 comprising three-electrodes, a working electrode (101), a counter electrode (102), and a reference electrode (103).
17. Apparatus according to clause 16 in which the three electrodes (101,102,103) are aligned.
18. Apparatus according to clause 16 in which the three electrodes (101,102,103) are forming a triangle.
19. Apparatus according to clause 16 in which the working electrode (101) and the counter-electrode (102) are made of platinum).
20. Apparatus according to any of clauses 16-19, wherein the three electrodes (101,102,103) are held in steady positions the one in respect to the other through being moulded in an acrylic resin holder (105).
21. Apparatus according to any of clauses 16-20 wherein the reference electrode (103) extends through a hollow tube (106) through the holder (105).
22. Method of any of clauses 1-8, wherein the sample comprises heavy metal ions dissolved in a bipolar solvent, such as water, and/or wherein the first frequency band is below 50 Hz and the second frequency band is between 1 kHz and 1 MHz.
23. Method of clause 22, wherein the heavy metals include Mercury (Hg), Cadmium (Cd), Arsenic (As), Chromium (Cr), Lead (Pb), Zinc (Zn), Copper (Cu), Iron (Fe), Silver (Ag) and Nickel (Ni) or other metals or metalloids showing atomic weights 63.5 and 200.6 gr/mol.
24. Method according to clause 22 or 23, wherein the ions comprise Zinc Sulfate and Lead Nitrate.
25. Method according to clause 24, wherein the concentrations of Zinc Sulfate and Lead Nitrate in water were measured at different temperatures such as 10, 20 and 30° C. and the different solutions were distinguishable for 5 ppm and 50 ppm.
26. Method according to any of clauses 22-25 wherein different ions are mixed in water and the concentrations are measured for each ion.
27. System comprising an apparatus according to any of clauses 9-21, configured to use a method according to any of clauses 1-8 and/or 22-26.

1-18. (canceled)

19. A system comprising an apparatus for measuring the concentration and presence of heavy metal ions (HMI) in a sample by electrochemical impedance spectroscopy (EIS), comprising:
a. a first module including a communication unit and configured to provide voltage and electrical currents in a first frequency band and to measure the impedance of the sample in the first frequency band,
b. at least a second module including a communication unit and configured to provide voltage and electrical currents in a second frequency band different from the first band and to measure impedance in the second band, and
c. a housing in which are arranged a system controller connected to the communication modules of the first and second module, a data processor, a power module, one or more environmental sensors, and a heating/cooling unit connected to a supply unit for providing cooling/heating fluid,
the data processor being configured to combine the measurements from the first frequency band with measurements in the second frequency band in said data processor, to calculate the concentration of a certain HMI and to establish in real time or near real time, wherein a priori knowledge is used by the data processor in the combination, and the a priori knowledge is related to measurements of certain samples comprising HMIs at earlier times, wherein HMIs are dissolved in a known solvent, such as water, and the concentrations are measured at a peak and a valley marking point in the Bode plot of the frequency bands to detect HMI in the known solvent.

20. The system according to claim 19, wherein the frequency bands comprise frequencies from 0.1 Hz-30 GHz, preferably 10-100 kHz, 100 kHz-1 MHz, and 1 Mhz-1 GHz, wherein the frequency is preferably swept over the different frequencies.

21. The system according to claim 19, wherein the sample comprises heavy metal ions dissolved in a bipolar solvent, such as water, and wherein the first frequency band is below 50 Hz and the second frequency band is between 1 kHz and 1 MHz.

22. The system according to claim 21, wherein the heavy metals include Mercury (Hg), Cadmium (Cd), Arsenic (As), Chromium (Cr), Lead (Pb), Zinc (Zn), Copper (Cu), Iron (Fe), Silver (Ag) and Nickel (Ni).

23. The system according to claim 19, comprising 12 modules each module functioning in a different frequency band, ranging from 0.1 Hz-10 GHz.

24. The system according to claim 19, wherein each module comprises a board provided with a temperature sensor connected to a secondary heating/cooling element for finely tuning the temperature control of the EIS module and the apparatus being provided with memory for storing data from earlier measurements, either locally or, preferably, remotely in a network with on-line access, and preferably provided with computer power for AI, or deep learning.

25. The system according to claim 19 comprising a working electrode, a counter electrode and a reference electrode.

26. The system according to claim 25 in which the three electrodes are aligned or in which the three electrodes are forming a triangle, in which the working electrode and the counter-electrode are preferably made of platinum.

27. The system according to claim 26, wherein the three electrodes are held in steady positions the one in respect to the other through being moulded in an acrylic resin holder, and the reference electrode extends through the acrylic resin holder in a hollow tube of larger diameter.

28. The system according to claim 19, wherein the a priori knowledge is stored in a library and the measurements in the first frequency band are compared with data in the library, whereafter it is decided which measurements are made in the second frequency band, if necessary.

29. The system according to claim 28, provided with an AI processor and an AI algorithm connected to the library, forming a processing unit.

30. The system according to claim 29, also including a number of measuring units provided on site and being connected to the processing unit through the Internet.

31. A method for measuring the concentration and presence of heavy metal ions (HMI) in a sample by electrochemical impedance spectroscopy (EIS), wherein:
a. the impedance of the sample is measured in a first frequency band of a first module including a communication unit and configured to provide voltage and electrical currents in the first frequency band,
b. the impedance is measured at least in a second frequency band of least a second module including a communication unit and configured to provide voltage and electrical currents in a second frequency band different from the first band, and the measurements from the first frequency band are combined with measurements in the second frequency band in a data processor, to calculate the concentration of a certain HMI and to establish in real time near real time), wherein a priori knowledge is used by the data processor in the combination, and the a priori knowledge is related to measurements of certain samples comprising HMIs at earlier times, wherein HMIs are dissolved in a known solvent, such as water, and the concentrations are measured at a peak and a valley marking point in the Bode plot of the frequency bands to detect HMI in the known solvent, and
c. the data processor is arranged in a housing, in which are also arranged a system controller connected to the communication modules of the first and second module, a data processor, a power module, one or more environmental sensors, and a heating/cooling unit connected to a supply unit for providing cooling/heating fluid.

32. The method according to claim 31, wherein the frequency bands comprise frequencies from 0.1 Hz-30 GHz, preferably 10-100 kHz, 100 kHz-1 MHz, and 1 Mhz-1 GHz, wherein the frequency is preferably swept over the different frequencies.

33. The method according to claim 31, wherein rinsing takes place after each or a number of measurements.

34. The method according to claim 31, wherein the sample comprises heavy metal ions dissolved in a bipolar solvent, such as water, and wherein the first frequency band is below 50 Hz and the second frequency band is between 1 kHz and 1 MHz.

35. The method according to claim 34, wherein the heavy metals include Mercury (Hg), Cadmium (Cd), Arsenic (As), Chromium (Cr), Lead (Pb), Zinc (Zn), Copper (Cu), Iron (Fe), Silver (Ag) and Nickel (Ni).

36. The method according to claim 35, wherein the concentrations of Zinc Sulfate and Lead Nitrate in water were measured at different temperatures such as 10, 20 and 30° C. and the different solutions were distinguishable for 5 ppm and 50 ppm, also when different ions were mixed in water and the concentrations are measured for each ion.
